(19) United States
(12) Patent Application Publication
(10) Pub. No.: US 2008/0319518 A1
(43) Pub. Date: Dec. 25, 2008

(54) CHARGED PARTICLE GENERATOR AND FUNCTIONAL FABRIC HAVING A CHARGED PARTICLE EMISSION FUNCTION

(75) Inventor: Masakazu Komuro, Setagaya-ku (JP)

Correspondence Address: OBLON, SPIVAK, MCCLELLAND MAIER & NEUSTADT, P.C., 1940 DUKE STREET, ALEXANDRIA, VA, 22314, US

(73) Assignee: NAC CO., LTD., Setagaya-ku (JP)

(21) Appl. No.: 11/852,539

(22) Filed: Sep. 10, 2007

(30) Foreign Application Priority Data
Oct. 25, 2006 (JP) 2006-290057
Jan. 29, 2007 (JP) 2007-017594

(51) Int. Cl. A61F 7/00; H01B 1/24; H01B 1/04

(52) U.S. Cl. 607/100; 252/500; 252/503; 252/507

(57) ABSTRACT

Provided are a charged particle generator that continually generates large quantities of charged particles as a result of temperature dependent excitation, functional fibers comprising composite carbon particles, having SP³ and SP² structures, and a healthcare device that can increase the effect of penetration of the human body by charged particles without limit in time, and which can produce a synergistic effect with infrared radiation. The charged particle generator comprises semiconductor particles and a coupling agent, blended at a ratio that results in a percolation effect, and is disposed at a human body contact face of the healthcare device, so as to achieve a body temperature elevating effect and blood circulation improving effect. The functional fibers can be used in processed or formed products for improved effects in the heating range produced by the human body, as well as in combination with a magnet, for a further synergistic effect.

### TECHNICAL FIELD

The present invention relates to a charged particle generator and to a functional fabric having a charged particle emission function. More specifically, the present invention relates to a healthcare device that is excited by body heat and generates infrared radiation and charged particles, but primarily charged particles, and to a healthcare device having the effect of penetrating the human body with these charged particles and infrared radiation.

The present invention also relates to functional fibers having an infrared radiation emitting function and a charged particle emitting function, and to processed products and formed products comprising this functional fabric. More specifically, it relates to a material wherein carbon particles having an SP³ diamond structure produced by using shockwaves and an outer coating of an SP² graphite structure are blended with fibers or affixed to fibers and to processed products and formed products including these functional fibers, which are primarily used in promoting and maintaining human health. The fibers to which the present invention is applied may be cotton, which comprises short fibers, or threads made from this cotton and, in the present invention, both are collectively referred to “fiber”.

### BACKGROUND ART

In order to take advantage of the blood circulation promoting effect of magnetic field on the human body, it is common for magnetic materials to be made into chips, which are affixed to the human body with adhesive tape or the like, and used as healthcare devices. Ferrite magnets with (BH)max values of approximately 3 and Alnico metal magnets with (BH)max values of approximately 5 to 10 are used for the magnets, and recently, high energy product rare earth magnets reaching (BH)max values as high as 10 to 30 are in use.

Infrared radiation has also been found to have a blood circulation promoting effect, a nerve fiber activating effect, an analgesic effect and the like. As with magnets, chips are fabricated and used as healthcare devices. Germanium and tourmaline are commonly used as infrared radiation emitting materials, but recently diamond semiconductors formed by shockwaves, which have excellent infrared radiation capacity in the 4 to 12 μm wavelength range have also been devised by the present inventors.

Charged particles, which are generated by activating piezoelectric/pyroelectric oxide materials such as tourmaline and single crystals such as germanium with body heat, are coming into use, as they have been found to have a muscle fatigue relieving effect and an analgesic effect as a result of penetrating the human body. Recently, because the actions and effects of magnetic field alone or infrared radiation alone are limited, composite magnets have been devised, comprising a magnetic material and an infrared radiation emitting material that has a piezoelectric/pyroelectric effect, so as to produce a synergistic effect from magnetic field, infrared radiation and charged particles (for example, see JP-05-347206-A).

However, in so far as concerns healthcare devices using the proposed piezoelectric/pyroelectric materials such as tourmaline and single crystal semiconductor materials such as germanium, as long as the heating effect that is caused by body heat continues, infrared radiation is activated, the infrared radiation inherent to the material is emitted, and the effect is maintained. However, germanium has a small band gap of approximately 0.6 eV, and infrared radiation released from the 0.01 eV donor level thereof is primarily in the 100 μm wavelength range. Infrared radiation at a wavelength of 100 μm, which is near that of infrared radiation released from extremely cold objects at approximately 30° K, has little heating effect.

Tourmaline emits infrared radiation at wavelengths of 4 to 10 μm, which has a great heating effect, but because this is an insulator, the number of carriers that emit infrared radiation with excitement at the body temperature levels is small, and therefore sufficient quantities of radiation cannot be ensured.

Furthermore, charged particles generated by the piezoelectric/pyroelectric effect are generated when the tourmaline is heated as a result of body heat and the crystal is subject to stress, or when changes in the temperature difference between body temperature and the healthcare device continue so that the crystal is subject to stress. Accordingly, after putting on the healthcare device, when the overall temperature becomes constant, because the piezoelectric/pyroelectric material is an electrical insulator, the amount of charge emitted is greatly reduced, so that the charged particle effect can no longer be expected. In other words, because the charge emission effect of the piezoelectric/pyroelectric material is limited to the period of time up to the point at which the healthcare device reaches a constant temperature, the time during which it is effective is limited. Furthermore, piezoelectric/pyroelectric materials such as tourmaline are electrical insulators, and therefore the number of charged particles is small, so that the charged particles that are generated, which are accelerated by an electrical field, have little mobility within the object, and little charged particle penetration effect can be expected.

For composite magnets, which are manufactured in order to achieve a synergistic effect from the effects of the charged particles, the infrared radiation and the magnetic field, and which are fabricated by press molding a mixture of a powdered magnetic material and a powdered infrared radiation emitting material, resin molding manufacturing methods have also been devised wherein powdered tourmaline, which is an infrared radiation and charged particle emitting material, is first given an insulating coating with a coupling agent, in order to particularly increase the effect of the charged particles (see JP-2001-126908-A).

Furthermore, with regard to the body penetrating effect of the charged particles, charge generation is limited to the time up to the point at which a constant state is reached, and if the surface of the tourmaline, which is the piezoelectric/pyroelectric material, has been insulated, because it is difficult for the charged particles to pass through the insulating film, almost no effect can be expected. Furthermore, because the lifetime and mobility of charged particles in the bonding resin is not great, even those charged particles that do pass through the insulating film become trapped in the resin, so that even if charges are generated from tourmaline, the amount that reaches the surface of the human body is small.

Germanium is a semiconductor and therefore has high charged particle radiation capacity, and because the wavelength of the infrared radiation resulting from the semiconductor band structure is long, at 100 μm, the heating effect resulting from the infrared radiation is small. Consequently, substantially no charge penetration effect can be expected with conventional composite magnets using infrared radiation and charged particles. Even if germanium emits charged particles as a result of heating at body temperature levels, with bulk germanium crystals, the electromotive force resulting from the Seebeck effect generated by the temperature difference resulting from heating at body temperature is no greater than approximately 1 mV, whereas the impedance of the human body is great, at several hundred Ω, so the effect of penetration of the human body by these charged particles is slight. Consequently, this cannot be expected to be effective as a healthcare device.

In order to take advantage of the blood circulation promoting effect of magnetic field on the human body, it is common for magnetic materials to be made into chips, which are affixed to the human body with adhesive tape or the like, and used as healthcare devices. In terms of magnets, Pt—Co metal magnets are used that have (BH)max values of 10 to 20, and recently, high energy product rare earth magnets reaching (BH)max values as high as 30 to 40 are in use.

Infrared radiation has also been found to have a blood circulation promoting effect, a nerve fiber activating effect, an analgesic effect and the like. As with magnets, chips are fabricated and used as healthcare devices. Infrared radiation emitting materials which are in use range from germanium, which releases far infrared radiation at wavelengths of approximately 100 μm, to ceramic materials such as tourmaline, which release infrared radiation at wavelengths of 10 to 15 μm, and oxides of metals such as titanium, some of which are used by way of blending them with fibers (for example, see JP-03-190990-A).

Furthermore, charged particles, which are emitted as a result of activating piezoelectric/pyroelectric materials such as tourmaline by body heat, are coming into use, as they have been found to have a muscle fatigue relieving effect and an analgesic effect as a result of penetrating the human body. Recently, because the actions and effects of magnetic field alone or infrared radiation alone are limited, composite magnets have been devised, comprising a magnetic material and an infrared radiation emitting material that has a piezoelectric/pyroelectric effect, so as to produce a synergistic effect from magnetic field, infrared radiation and charged particles (see JP-2001-126908-A).

The present inventors proposed a surface coated composite magnet that takes advantage of the action at a distance effects of magnetic field, and the action through a medium effects of infrared radiation and charged particles in JP-2006-042915-A, but even in this surface coated composite magnet, the charge penetration effect was limited in time. For this reason, there was a demand for a material producing large amounts of infrared radiation and charged particle emissions, and more specifically for a healthcare device that generates large amounts of charged particles as a result of heating at body temperature, and which produces a great penetration effect.

With fibers using proposed infrared radiation emitting materials, such as alumina, titanium and colloidal platinum, as long as the heating effect that is caused by body heat continues, infrared radiation is activated, the infrared radiation inherent to the material is emitted, and the effect is maintained. Because almost all of the infrared radiation emitting materials used are inorganic insulating materials, the band gap is large, and few carriers are excited by heating at body temperature levels, and therefore the infrared spectrum emission rates are low at wavelengths of 4 to 15 μm, which are the most important for healthcare devices. Infrared radiation at wavelengths of 4 to 15 μm has the greatest heating effect on the human body. Furthermore, very small amounts of charged particles are generated as a result of the infrared radiation emitting material being heated by body heat and the crystal being subject to stress; and because this material is an electrical insulator, the emitted charge is greatly attenuated, so that the charged particle effect can no longer be expected, and thus the effect is limited in time.

Furthermore, in terms of the infrared spectral characteristics of the materials that are blended with fibers, in examples such as that recited in JP-05-347206-A, the temperature was 700 to 1300° K, which is greatly removed from actual body temperatures, and there are no data in terms of characteristics when actually used, so this cannot be termed an effective material. Thus, there is a great need for materials with which the infrared radiation and charged particle emission effects produced as a result of excitement by heating at temperatures in the vicinity of body temperature, but materials satisfying this requirement have not yet been produced.

Furthermore, the fibers that are blended, and the adhesive that affixes the infrared radiation emitting powder to the fiber surfaces are polymers, and as the functional groups associated with the basic main chains of the polymer have great infrared absorption capacity, the infrared radiation, which is emitted from infrared radiation emitting materials at the interior of the fibers, or at the interior of the adhesive, as a result of activation of the infrared radiation emitting material by body heat, is absorbed within the material and does not readily reach the surface of the fiber. Consequently, the blended infrared radiation emitting material is not effectively used, and thus there has been an increasing demand for materials having a large capacity for emitting infrared radiation and for emitting charged particles at temperatures ranging from room temperature to the vicinity of body temperature.

Furthermore, in terms of the effects of penetrating the human body by charged particles, even though some charge was generated from conventional metal oxide type infrared radiation materials, the time until a constant state was reached was limited, and therefore almost no effect could be expected. Furthermore, because the lifetime and mobility of charged particles in polymer fiber materials and bonding resins is not great, they tend to be trapped before escaping, and if the amounts emitted are not large, even if charges are generated, the amount that reaches the surface of the human body is small. Thus, in these terms as well, almost no charge penetration effect on the human body can be expected from fibers to which the conventional infrared radiation emitting materials have been admixed or affixed with bonding resins.

The present invention is particularly directed at improving the charged particle penetration capacity of the conventional composite healthcare devices described above. In other words, a first object of the present invention is to provide a charged particle generator that continually generates large quantities of charged particles as a result of temperature dependent excitation. Furthermore, a second object is to provide a healthcare device that fully achieves a synergistic effect from infrared radiation and charged particles by continuously penetrating the human body with charged particles when the healthcare device is being worn, without the effect of the charged particles penetrating the surface of the human body being limited in time.

The present invention is directed at increasing the infrared radiation and the charged particle penetration effect of fibers with which the aforementioned conventional infrared radiation materials have been blended or fibers to which the aforementioned infrared radiation materials have been affixed with bonding resin. That is to say, an object of the present invention is to provide a functional fiber and a processed product or formed product thereof that can be used as a material for a healthcare device which fully achieves a synergistic effect from infrared radiation and charged particles by continuously penetrating the human body with charged particles and infrared radiation, preferably in conjunction with magnetic field, when the healthcare device is being worn on the body, or when in contact with the body, without the effect of the charged particles and infrared radiation penetrating the surface of the human body being limited in time, which is a fiber produced by a predetermined process, or a processed product (thread, fabric or the like) comprising this fiber.

### DISCLOSURE OF THE INVENTION

In order to achieve the first object as described above, the charged particle generator according to the present invention comprises a semiconductor powder blended and formed with an electrically insulating coupling agent, at a semiconductor powder blending ratio of 3 to 20 vol % with respect to the coupling agent, so that primarily charged particles are generated as a result of temperature dependent excitation (claim 1).

When semiconductor powder is blended and formed with an electrically insulating coupling agent, if the semiconductor powder blending ratio is within the range of 3 to 20 vol %, a situation results wherein the semiconductor powder is internally connected in series (percolation), which greatly lowers electrical resistance, allowing for a large electromotive force. This is an effect (percolation effect) resulting from series connection of the semiconductor powder in the coupling agent. When semiconductor powder, which is in the state allowing for percolation, is subject to temperature gradient excitation, because the surrounding coupling agent is an insulator, the charged particles generated by the semiconductor powder are transmitted between series connected particles, whereby charges exist from the surface of the charged particle generator.

The percolation effect is dependent on the semiconductor powder particle size, and normally occurs with blending in the range of 3 to 20 vol %, and is most effective at 5 to 7 vol % (claim 2). The size of the semiconductor particles is preferably within the range of 5 nm to 1 μm. At less than the 5 nm, the semiconductor band structure is disturbed, and if 1 μm is exceeded, there is a large degree of electrical repulsion between the excited charged particles and within the particles. The charged particles are no longer capable of one-dimensional movement, and the potential difference within the particle is lowered. In bulk semiconductors, thermally excited charged particles move in three dimensions, and as a result the potential generated by the element drops.

When the semiconductor is finely powdered, blended with an electrically insulating organic resin or coupling agent comprising an inorganic glass and formed, by adjusting the amounts of the semiconductor powder and the coupling agent that are blended so as to be within the range of 3 to 20 vol %, an electrical percolation effect can be created which results in the provision of a large electromotive force. As a result of heating this charged particle generator, for example, by way of contact with the human body, charged particles are generated due to the potential gradient with the human body, and the human body can be penetrated thereby.

When the charged particle generator described above is used in a healthcare device, the charged particle generator is provided at the human body contact face of the healthcare device. When this healthcare device is used by wearing it on the body, as a result of the heating effect due to body heat at the human body contact face, and the cooling effect due to the metallic band or the nonmetallic band comprised by the healthcare device, a temperature difference is generated between the particles, so that emission of the infrared radiation and the charged particles is maintained while the healthcare device is worn.

Semiconductor having an energy level of no greater than 0.5 eV are such that, even in the case of bulk semiconductors, the semiconductor is excited and generates charged particles as a result of heating at body temperature, and a carrier density difference occurs within the semiconductor on the basis of the temperature differential, which generates electrical potential. This is known as the Seebeck effect (claim 8).

In this case, the potential gradient resulting from the generated charge works towards homogenizing the carrier density, due to the repulsion between the charges. Consequently, the potential difference between the two ends of a bulk semiconductor is low, and even with a high purity semiconductor with a carrier density of 10E16, the Seebeck constant is only at several hundred μV (Thermoelement, The Nikkan Kogyo Shimbun, Ltd., written by Joffe, translated by Sakata, pp 58, FIG. 2.11).

Preferably, semiconductor carbon particles having an SP³ and SP² composite structure formed by shockwaves are used for the semiconductor powder (claim 3). Furthermore, it is preferable that epoxy resin or low melting point glass powder or the like be used for the coupling agent comprising an electrically insulating organic resin or inorganic glass (claim 4).

Semiconductor carbon powder particles having an SP³ and SP² composite structure formed by shockwaves are mixed with epoxy resin or low melting point glass powder or the like and extruded to form the charged particle generator. Next, this is embedded in a metallic band or nonmetallic band serving as a human body contact part, or the charged particle generator is affixed to the top face of a magnet and/or piezoelectric/pyroelectric element, which is embedded in the metal band or nonmetallic band beforehand, so as to complete the healthcare device according to the present invention. Persons wearing this healthcare device simultaneously receive the effects of the magnetic force lines, the charged particles and the infrared radiation.

The following two methods are specific examples of methods for producing the semiconductor composite carbon particles having a SP³ and SP² structure formed by shockwaves. (1) A method wherein high-performance CB explosive is exploded in a sealed container so as to momentarily produce 2,000,000 atmospheres of pressure and several thousand degrees of heat, so as to form semiconductor carbon particles having a composite structure. (2) A method wherein fine carbon powder, copper powder and the like are placed in a vessel and an explosive, which is placed thereupon, is ignited, so as to apply similar pressures and temperatures to the mixed powder product and change the crystalline structure of the carbon to that of diamond semiconductor, whereafter the metal powder is dissolved with acid to produce particles having the structure described above. (Eiji Osawa, Japan Nanonet Bulletin 108 2006.03.08, Sumitomo Coal Mining KK, Cluster Technology Society, 06.03.27).

Diamonds, which have a high resistivity near that of an insulator, have a low impurity level density, so that charges are not readily sufficiently excited to jump the band gap as a result of heating at the body heat level. Consequently, there is no charged particle emission effect, and the amount of radiated light that is emitted when the excited charged particles fall back into the valance band is also small. Conversely, during the particle production process, impurities, and particularly nitrogen contained by the explosive, are included in semiconductor composite carbon particles that are produced by the shockwave method, so that these tend to act as an N-type semiconductor; furthermore, as a result of stress within the particles and the like, due to the pressure at the time of the explosion, the solid band structure is disturbed so as to produce an impurity level of 0.2 to 0.40 eV, making it electrically conductive. Furthermore, because the powder has a particular crystal type of SP³ and SP² composite structure, the infrared radiation capacity and charged particle emission capacity thereof is five to ten times greater than that of conventional tourmaline or the like. Normal single crystal diamonds are almost perfect insulators, having a band gap of 5.5 eV and a resistivity of 10E16Ω at normal temperatures, but the SP³ and SP² composite carbon particles used in the present invention allow for resistivity values of approximately 10Ω to 10E10Ω, depending on manufacturing conditions.

Semiconductor that have been caused to have an activation energy level of no greater than 0.5 eV by way of an additive are preferred because they generate large amounts of carriers that are thermally excited at body temperature levels. Specific examples include, diamonds formed by shockwaves, germanium and silicon, which are single element semiconductors, InSb, BiTe and PbTe, which are compound semiconductors, Ca—Mn, Ca—Cr, Zn and Ti oxides, which are oxide semiconductors, FeSi₂ and CoSi, which are silicide semiconductors, and the like. Particles of one of these, or of a plurality of these, can be used as the semiconductor powder (claim 7).

When the semiconductor particles create a percolation effect in a formed insulator body (charged particle generator), the semiconductor particles are electrically series connected, but are arranged in a configuration in which they are insulated from each other in the lateral direction. Because the semiconductor particles are fine, the amount of charge generated in the individual particles is small, and no repulsion is generated internally like as in the manner of bulk semiconductors, and because the potential is series connected between the particles, the cumulative potential generated in the formed body is great, and a voltage of several volts is generated. It will be understood that the electromotive force of the charged particle generator according to the present invention is dramatically greater than that of conventional bulk germanium crystals, with which, as described above, the electromotive force resulting from the Seebeck effect, which is produced by the temperature difference resulting from heating at body temperature, is no greater than approximately 1 mV.

The internal impedance of the charged particle generator according to the present invention is higher than that of a bulk semiconductor, which is advantageous in that organisms can be more effectively penetrated by the charge because of impedance matching, the impedance of organisms being high, at several hundred Q (claim 1). In the healthcare device, if use is made of the synergistic effect of infrared radiation and magnetic field, in addition to the charged particle penetration effect, the therapeutic effect on the body can be increased.

If an insulating oxide magnetic powder is added to the coupling agent, a healthcare device can easily be produced with which, in addition to the charge penetration effect, a synergistic effect can be expected from the magnetic field and the infrared radiation, thus further increasing the effect on the human body.

In order to achieve the object described above, the present invention is a functional fiber wherein composite carbon particles having an SP³ diamond structure and an SP² graphite structure formed by shockwaves (hereinafter also referred to simply as composite carbon particles) are blended with or affixed to fibers, so as to have an infrared radiation emitting function and a charged particle emitting function (claim 10).

The functional fiber according to the present invention is cotton comprising short fibers wherein some or all of those short fibers comprise functional short fibers with which the composite carbon particles have been mixed or to which the composite carbon particles have been affixed (claim 11).

Composite carbon particles formed by shockwaves, having a structure wherein semiconductor diamonds having the SP³ structure are covered with an electro-conductive SP² graphite carbon film, are mixed with the fibers or affixed to the surface of the fibers with bonding resin. These fibers are processed so as to spin threads or so as to make non-woven fabrics or cloth (fabrics) and so as to further produce formed products, which are used to manufacture finished products such as strings, bands and belts, and when worn in contact with the human body, these are capable of producing an infrared radiation effect and a charged particle penetration effect in the human body (claims 12, 13).

Depending on the infrared wavelength band required, a composite powder wherein the composite carbon particles are blended with one type of powder or a plurality of types of powders selected from other infrared radiation materials, such as silica, germanium, compound semiconductors and metal oxides such as tourmaline, may be blended with or affixed to the fibers, so as to produce a functional fiber having an infrared radiation emitting function and a charged particle emitting function (claim 14).

Preferably, the composite powder described above comprises no less than 2 wt % of the composite carbon particles (claim 15). Next, the functional fiber having the infrared radiation emitting function and the charged particle emitting function, as a result of the composite powder, in which the other powdered infrared radiation emitting material is admixed to the composite carbon particles, being blended with or affixed to the fibers, is processed so as to make a functional thread or functional cloth, and the processed product or formed product resulting therefrom can be used as a healthcare device (claim 16 and 17).

### BRIEF DESCRIPTION OF THE DRAWINGS

FIG. 1 is a graph showing the electrical resistance characteristics of a product wherein the semiconductor particles and epoxy resin are blended, in one mode of embodiment of the charged particle generator according to the present invention.

FIG. 2 is a schematic view illustrating an application of the charged particle generator of the present invention to a healthcare device.

FIG. 3 is a graph showing the temperature characteristics of a product wherein the diamond semiconductors and a resin are blended.

FIG. 4 is a graph showing the temperature characteristics of a product wherein InSb semiconductors and a resin are blended.

FIG. 5 is an infrared spectral radiance graph for the composite carbon particles and tourmaline.

FIG. 6 is an infrared spectral radiance graph for conventional functional fibers in which tourmaline is blended at 10 wt %.

FIG. 7 is an infrared spectral radiance graph for functional fibers according to the present invention.

### DETAILED DESCRIPTION OF THE INVENTION

According to the first aspect of the invention, the charged particle generator comprises a semiconductor powder that is blended and formed with an electrically insulating coupling agent, at a blending ratio of 3 to 20 vol %, whereby the semiconductor powder in the charged particle generator is internally connected in series, which greatly lowers electrical resistance and produces a percolation effect, resulting in a large electromotive force, so that an extremely large quantity of charged particles is emitted as a result of thermal excitation.

According to the fourth aspect of the present invention, the semiconductor particles that are blended at 3 to 20 vol % in resin or glass bond material are such that the potential between the particles that are series connected is cumulative and a greater electrical potential is generated than with bulk semiconductors. Furthermore, as compared to bulk semiconductors, the particles are easier to form, less expensive and offer more choices in terms of shape. Because these particles are used by affixing them to the surface of a healthcare device, there is a great degree of freedom in terms of the shapes of devices with which these can be used.

According to the fifth aspect of the present invention, because the semiconductor powder is blended with resin or glass bond and integrally formed, the mass production characteristics are improved.

According to the seventh aspect of the present invention, specific ordinary semiconductors that can be powdered can be used, which facilitates manufacturing.

According to the eighth aspect of the present invention, because all powdered semiconductor particles having a band structure with an activation energy level of no greater than 0.5 eV can be used, the choice of semiconductors is not restricted and the cost is lowered. Furthermore, as compared with bulk semiconductors, a small amount of semiconductor raw material is sufficient, which is highly advantageous in terms of cost.

According to the ninth aspect of the present invention, the healthcare device is such that the charged particle generator is disposed at the human body contact face of a metallic band or a nonmetallic band, and therefore by using the percolation effect of the semiconductor particles, the electrical potential generated is great, and the effect of penetration of the human body by the charged particles is great. Next, when the healthcare device is worn, the charged particle penetration effect and the infrared radiation effect can be expected from the semiconductors at all times, so that the effect of increasing body temperature is dramatically greater than with conventional products.

According to the 10th aspect of the present invention, a functional fiber having an infrared radiation capacity and a charged particle emission capacity at body temperature or in the vicinity of room temperature comprises composite carbon particles which are admixed with the fibers or affixed to the surface of the fibers. By using the infrared radiation emission and the charged particle emission resulting from the impurity level excitation as a result of body heat and the charged particle penetration effect that is based on the electrical field generated by the temperature difference between the particles, as differs from conventional fibers to which infrared radiation emitting powders have been admixed, healthcare devices employing this functional fiber maintain their effects at all times when worn on the human body.

According to the 11th aspect of the present invention, healthcare devices using the fibers can be provided in the form of pillows and cushions. According to the third aspect of the present invention, an effect can be produced where required on the surface of, or within, the human body in the shape of dots, lines or clusters, by way of infrared radiation and charged particle emission, using body heat as an energy source, thus allowing for the provision of a novel material which can be used for health promotion and/or therapy.

According to the 14th aspect of the present invention, it is possible to provide functional fibers that emit charged particles and infrared radiation in the necessary wavelength band, and healthcare devices using these fibers. Furthermore, because infrared radiation emitting particles, which deliver different wavelengths than the composite carbon particles, can be used in conjunction therewith, it is possible to broaden the infrared wavelength band and to reduce the cost of the manufactured product.

According to the 13th and 17th aspect of the present invention, healthcare devices can be provided which have been processed and shaped into desired forms, such as necklaces, bracelets, rings, anklets, undergarments, socks, stomach bands, sheets, pillows and bedclothes or flooring covering such as carpets. These devices make use of the combined effect on the human body of the charged particle penetration effect and the infrared radiation penetration effect, by employing the infrared radiation emission capacity and the charged particle emissions resulting from body temperature heating of the diamond semiconductors.

### BEST MODE FOR CARRYING OUT THE INVENTION

In order to increase the charged particle penetration effect and the infrared radiation effect from the semiconductor particles, a semifinished product, which is a liquid in which electrically insulating resin bond or glass bond and the semiconductor powder are mixed, is poured into an extrusion molder and subjected to a hot press-molding process in a predetermined shape, so as to produce, as a formed product, a charged particle generator. These charged particle generators are fixed in place with adhesive or the like, in holes that have been formed in a metal band, a requisite number of these being arranged so as to complete the healthcare device. The charged particle generator used in the healthcare device is preferably disposed so as to be in close contact with the human body, so that the charged particles that are excited by way of activation by body heat generate electrical potential, and thus produce the charged particle penetration effect. Consequently, it is possible to produce a continuous charged particle penetration effect and infrared radiation effect from the semiconductor.

Next, modes of embodiment of the present invention will be described. The actual method of manufacturing composite carbon particles, having an SP³ diamond structure and an SP² graphite structure, formed by shockwaves, is already known. Specific examples include exploding a high-performance CB explosive in a sealed container so as to momentarily generate pressures of 2,000,000 atmospheres and temperatures of several thousand degrees, so as to form UDD (ultra dispersed diamond), or placing fine carbon powder, copper powder and the like in a sealed container, and igniting explosives that have been placed on top of these, so as to subject the powder mixture to similar pressures and temperatures, and after turning the carbon into diamond, dissolving the metal powder with acid so as to produce particles having the structure described above.

The composite carbon particles are electrically conductive, readily producing impurity levels of approximately 0.1 to 0.3 eV as a result of impurities such as the nitrogen from the explosives and stresses within the particles resulting from the manufacturing process. Because the powder has a special SP³ and SP² composite structure, the infrared radiation emission capacity and charged particle emission capacity are 5 to 10 times greater than those of tourmaline and the like, which are conventional metal oxide infrared radiation materials.

Normal diamonds are almost perfect insulators, having a band gap of 5.5 eV and a resistivity of 10E16Ω at normal temperatures, but the composite carbon particles used in the present invention allow for resistivity values of approximately 10Ω to 10E10Ω, depending on manufacturing conditions. Single crystal diamonds have a high resistivity near that of an insulator, so that charges are not readily sufficiently excited to jump the band gap as a result of heating at body heat levels. Consequently, there is no charged particle emission effect, and therefore the radiated light that is emitted when the excited charged particles fall back into the valance band is also small.

If the fiber is a synthetic fiber, it is preferable that the composite carbon particles be mixed in with the fiber during the spinning process so as to manufacture the threads, and in the case of natural fibers such as cotton or wool, it is preferable that the composite carbon particles be affixed to the surface of the fibers with bonding resin. Examples of synthetic fibers include nylon, vinylon, esters, acrylics, urethanes and the like. When such fibers are caused to carry the composite carbon particles, then processed so as to produce a healthcare device such as described above, which is used by wearing it on the human body, as a result of the heating effect of body heat at the human body contact face thereof, temperature differences occur within the particles and between the particles, so that when the healthcare device is worn, an effect is continually maintained wherein infrared radiation and charged particles from the semiconductor diamonds are emitted. The 4 to 10 μm infrared radiation resulting from the 0.1 to 0.3 eV impurity level of the semiconductor diamonds matches the radiation wavelengths for a black body heated to 300 to 400° C., and has a large heating effect on the human body. The composite carbon particles that are manufactured by the shockwave method have an SP³ diamond structure that tends to include nitrogen from the explosives and the like used in the manufacturing process, resulting in a 0.1 to 0.3 eV impurity level in the diamond band gap. The carriers excited in this impurity band emit infrared radiation with a wavelength of 4 to 15 μm when the energy is gone.

A preferred embodiment of the present invention is produced by kneading composite carbon particles having an SP³ and SP² structure, formed by shockwaves, with a synthetic fiber material at a predetermined ratio and spinning this. In the case of natural fibers, the surfaces of the fibers are coated with a mixture in which composite carbon particles and bonding resin are mixed, by dipping or spraying during the thread making finishing process, and this is cured to affix the mixture to the surfaces of the fibers. This affixing method can also be applied to synthetic fibers. In cases where the composite carbon particles are affixed to the surface of the fibers, this may be achieved by blending the infrared radiation emitting material blended at a predetermined ratio with, for example, an aqueous acrylic resin emulsion adhesive, stirring so as to form a dispersion, causing this to adhere to the surface of the fibers by dipping or spraying, and thermosetting. In certain cases, the composite carbon fibers may be affixed to the fiber surface according to the method described above, after the forming process has been performed.

Furthermore, in cases where the functional fiber provided with the composite carbon particles of the present invention is supplied for use in a healthcare device, it is preferable that a magnet be disposed at the back of these functional fibers, or at the back of the processed product or formed product that comprises these fibers, so as to achieve a synergistic effect on the human body by way of the magnetic field, the infrared radiation and the charged particles. If these are disposed at the back of a magnetic material, a combined effect can be expected from the infrared radiation, the charged particles and the magnetic field. Furthermore, a magnetic oxide powder such as ferrite may be used as a means for achieving the magnetic field effect, by mixing it with the composite carbon particles and processing these together. Thus, by making use of, in addition to the charged particle penetration effect, the synergistic effects of infrared radiation and magnetic field, the healthcare effects on the body can be further improved.

In cases where the composite carbon particles are mixed with the synthetic resin material and this is spun, the particles will not readily come out as a result of mechanical abrasion when worn on the body or as a result of washing or the like, but the charged particle and infrared radiation effects from the particles that are present within the fibers will be diminished in accordance with the diameter of the fibers. The opposite is true in cases where the composite carbon particles are affixed to the surface of the fibers.

The functional fibers comprising the admixed or affixed composite carbon particles are processed into string, cloth, non-woven fabric or the like, and formed into objects that are worn or used in contact with the human body so as to be heated by body heat, such as for example bracelets, necklaces, stomach bands, clothing, socks, pillows, carpets, belts and the like. In certain cases, the functional fibers may be used by composite spinning with fibers produced by conventional processes.

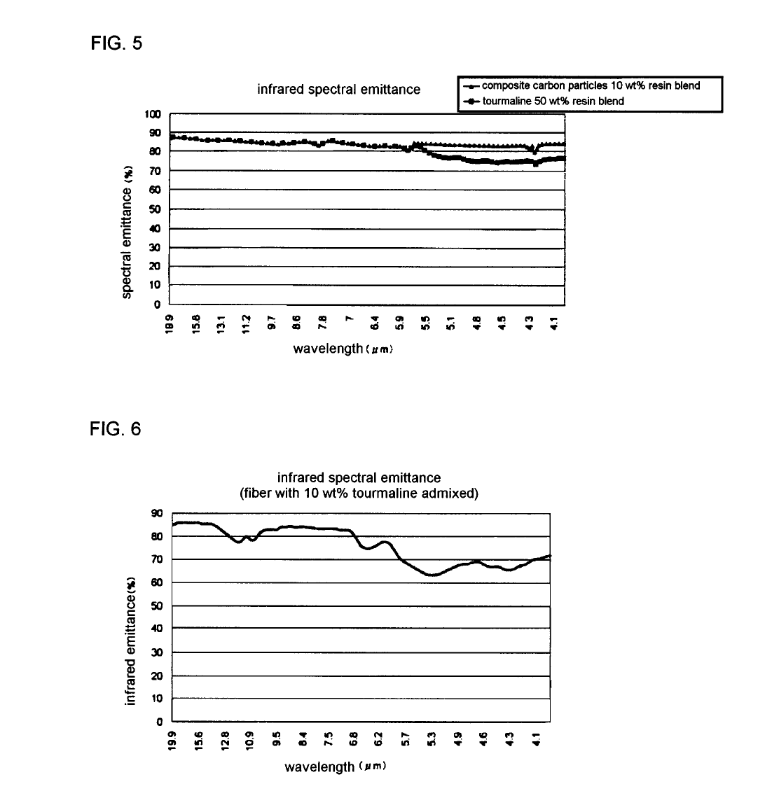

FIG. 5 comparatively illustrates the infrared spectral radiance characteristics of a product wherein a bonding resin and the composite carbon particles used in the present invention are mixed, and a product wherein bonding resin and tourmaline, which is an infrared radiation emitting material used in ordinary healthcare devices, are mixed. When the amount of tourmaline in the blend was increased by 5 times, the infrared radiation emission capacity at a wavelength of 7 μm or more was approximately the same as with the composite carbon particles, but in the band below 7 μm, this was lower than with the composite carbon particles. It is understood that the effect resulting from semiconductor excitation of the composite carbon particles is such that the infrared radiation characteristics in the 4 to 10 μm wavelength band are excellent.

### EXAMPLES

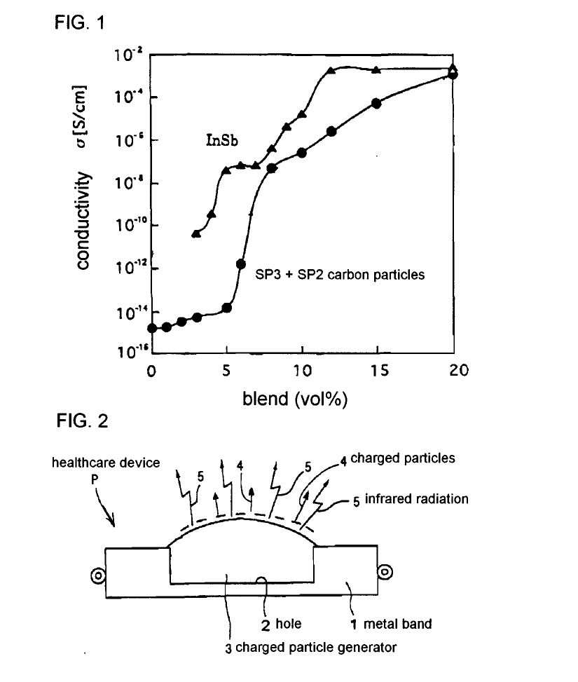

The relationship between the quantity of semiconductor particles in the blend and electrical conductivity of the charged particle generator having the percolation effect with the semiconductor particles of the present invention is shown in FIG. 1. A powder of SP³ and SP² composite carbon particles (diamonds) formed by shockwaves, having an average particle size of 20 nm, and an InSb powder having an average particle size of 0.5 μm were used for the semiconductor powders. The semiconductor particles were blended with epoxy resin at various percentages by volume, and pressure formed in a predetermined shape with an extrusion molder, so as to form pellets (charged particle generators). In all of these, the conductivity changed suddenly in the vicinity of 5 to 15 vol %, showing that percolation alignment was achieved in the vicinity of 5 to 9 vol %.

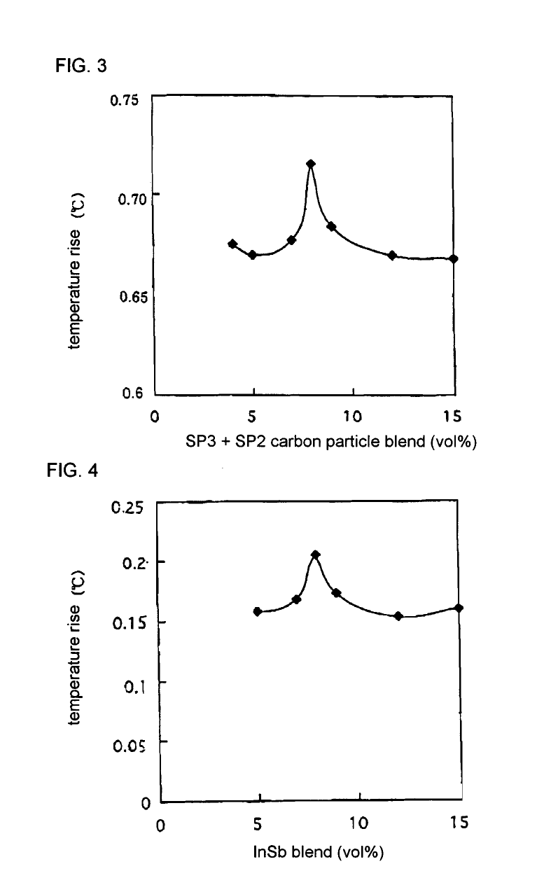

FIG. 2 is a sectional view of a healthcare device P, produced by embedding ten charged particle generators 3 in holes 2 of a metal band 1. The charged particle generators 3 are produced by blending the semiconductor particles mentioned above (SP³ and SP² composite carbon particles and InSb powders) with epoxy resin at various percentages by volume, and processing so as to form these at 4 mmφ×3 mmt. The temperature rise after 10 minutes when the healthcare device P was worn on the human body was measured with a thermograph. The measurement values are shown in FIG. 3 and FIG. 4. In the blending range at which percolation alignment was achieved, the rise in body temperature was great, indicating that this was effective as a healthcare device.

The surface potential of the charged particle generator that is heated to body temperature, in which SP³ and SP² composite carbon particles are blended with resin at 7 vol %, showed a value of 30 to 60 V. Conversely, InSb was at 5 to 20 V. (The measurement equipment used was a Trek Electrometer, Model 344, with a 6000 B8 probe. The electrometer probe spacing on the sample surface was 1 mm.) In the case of a similarly structured healthcare device in which 2 mm square by 4 mmt bulk InSb crystal was embedded, almost no temperature rise could be seen, and the generated electrical potential was 80 to 150 μV. The healthcare device of the present invention, which comprises the charged particle generator having the percolation alignment, generates a large electromotive force, and has a dramatically greater effect on organisms than conventional healthcare devices using bulk semiconductor germanium and tourmaline.

The infrared spectral radiance was measured for a non-woven fabric of processed thread in which nylon 6 fibers were mixed with 10 wt % tourmaline, which is a conventionally used metal oxide infrared radiation emitting material, as well as at different blending ratios of tourmaline, and for a non-woven fabric of processed thread in which nylon 6 fibers were mixed with 10 wt % of the composite carbon particles according to the present invention at an environmental temperature all 40° C. using measurement equipment produced by the Japanese Chemical Testing Society and JEOL.

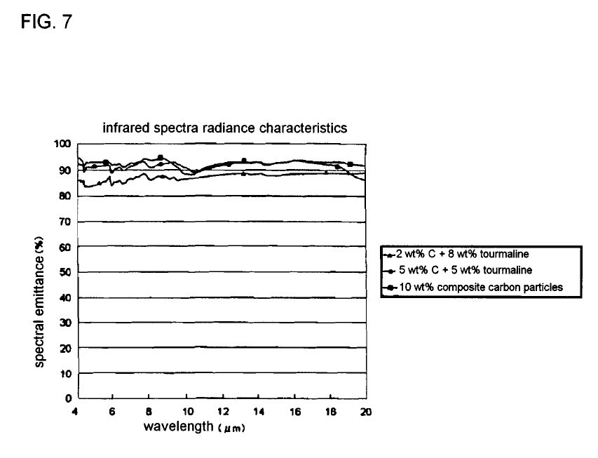

FIG. 6 shows the spectral characteristics of conventional functional fibers, which is to say, fibers blended with tourmaline, and FIG. 7 shows the spectral characteristics of functional fibers according to the present invention, which is to say, fibers to which the composite carbon particles were admixed.

Three types of fibers to which the composite carbon particles were admixed were measured. These comprised: 10 wt % of the composite carbon particles; 5 wt % of the composite carbon particles and 5 wt % of tourmaline; and 2 wt % of the composite carbon particles and 8 wt % of tourmaline. The size of the tourmaline particles used was no greater than 1 μm and the size of the composite carbon particles used was 0.1 to 0.01 μm. The nylon 6 polymer raw material and a predetermined infrared radiation emitting material powder were mixed and dissolved in a hexafluoro-2-propanol/dichloromethane solvent to produce a polymer concentration of 10%, this was extruded from a nozzle at a heating temperature of 225° C., to produce a dry spun fiber, which was made into a non-woven fabric.

From the results of the measurement, it was understood that the functional fiber wherein the composite carbon particles of the present invention were blended, had much better radiation characteristics, in all of the measured wavelength regions, than the conventional product and, in particular, in the 4 to 10 μm wavelength interval, the radiation characteristics were as much as approximately 20% better. These radiation characteristics are based on excitation of the diamond impurity level in the composite carbon particles at 40° C., which was an effect that could not be achieved with the conventional product. It is disadvantageous for the composite carbon particles to be admixed at a ratio of less than 2 wt %, as the charged particle penetration effect is inferior. Furthermore, the infrared radiation emission capacity is also reduced.

In cases where a magnet generating an external magnetic flux density of less than or greater than 400 G is used in combination with a formed product using the functional fibers with which the composite carbon particles of the present invention have been blended, it is possible to provide a healthcare device wherein, in addition to the infrared radiation effect and the charged particle penetration effect, a synergistic effect with the effect of the magnetic field can be expected, which has an even better health promotion and maintenance effect on the human body. Even in cases where this is used with pacemakers or the like, which must exert a weak magnetic field on the human body, there is no risk of adverse effects and the synergistic effect can be benefited from without worry.

### INDUSTRIAL APPLICABILITY

The healthcare device of the present invention generates a large electrical potential, and effectively penetrates the human body with excited charged particles as a result of heating the semiconductor particles at body temperature which, in combination with the infrared radiation emission capacity from the semiconductor powder, produces a synergistic effect. Consequently, in addition to using this healthcare device by forming it into necklaces, bracelets, rings, anklets, undergarments, socks, stomach bands, sheets, cushions and pillows, as desired, the present invention can be used as a veterinary healthcare device. This is particularly useful in industry, because it can be used even with electronic healthcare devices with which the use of magnet is not allowed, such as pacemakers, for example. The charged particle generator of the present invention has great thermoelectric power and therefore, in addition to healthcare devices, can also be used in sensor units such as fire alarms.

As is clear from the measurement results described above, functional fibers that make use of the infrared radiation and charged particle emission effect resulting from body heat and products formed therefrom have a greater effect than conventional products. Accordingly, when used in healthcare devices such as undergarments, support socks and the like, a muscle fatigue relieving effect can be expected as a result of improved blood circulation. Furthermore, the effect can be further improved by combining the synergistic effect of magnetic field and infrared radiation.

The healthcare device, which makes use of the combined effect on the human body of the charged particle penetration effect and the infrared radiation penetration effect of the functional fibers using the infrared radiation emission capacity and the charged particles that result from heating the diamond semiconductors of the present invention at body temperature, can be used by processing and shaping this into desired forms, such as necklaces, bracelets, rings, anklets, bands, belts, undergarments, socks, stomach bands, sheets, pillows and bedclothes as well as flooring covers such as carpets. In addition, it can also be applied to veterinary healthcare devices.

1. A charged particle generator comprising a formed mixture of semiconductor powder in an electrically insulating coupling agent, blended at ratio of 2 to 20 vol % of semiconductor powder with respect to the coupling agent, and generating primarily charged particles as a result of temperature gradient excitation.

2. The charged particle generator recited in claim 1, wherein the semiconductor powder is blended at a ratio of 5 to 7 vol % with respect to the coupling agent.

3. The charged particle generator recited in claim 1 or 2, wherein the semiconductor powder comprises composite carbon particles having SP³ and SP² structures formed by shockwaves.

4. The charged particle generator recited in claim 1, 2 or 3, wherein the electrically insulating coupling agent is selected from organic polymers and inorganic glass.

5. The charged particle generator recited in claim 4, wherein the organic polymer is selected from epoxy, acrylic and carbonate resins.

6. The charged particle generator recited in claim 4, wherein the inorganic glass is low melting point glass.

7. The charged particle generator recited in any one of claims 1 to 5, wherein the semiconductor powder is of any one type or a plurality of types selected from diamonds formed by shockwaves, germanium and silicon, which are single element semiconductors, InSb, BiTe and PbTe which are compound semiconductors, Ca—Mn, Ca—Cr, Zn, and Ti oxides, which are oxide semiconductors, and FeSi2 and CoSi, which are silicide semiconductors.

8. The charged particle generator recited in any one of claims 1 to 7, wherein the semiconductor powder is a semiconductor powder having an activation energy level of 0.5 eV or less.

9. A healthcare device comprising a metallic or non-metallic band and the charged particle generator recited in any one of claims 1 to 8, the charged particle generator being provided on the side of the band that contacts a human body.

10. A functional fiber comprising a fiber and composite carbon particles, having an SP³ diamond structure and an SP² graphite structure, formed by shockwaves, which are mixed with or affixed to the fiber, and having infrared radiation function and charged particle emission function.

11. The functional fiber recited in claim 10, wherein the fiber is cotton comprising short fibers wherein some or all of these short fibers comprise functional short fibers with which the composite carbon particles having an SP³ diamond structure and an SP² graphite structure formed by shockwaves have been mixed or to which the composite carbon particles having an SP³ diamond structure and an SP² graphite structure formed by shockwaves have been affixed.

12. A functional thread made using the functional fiber recited in claim 10 or 11.

13. A processed product or formed product comprising the functional fiber recited in claim 10 or 11 or the functional thread recited in claim 12.

14. A functional fiber comprising a fiber and a composite powder in which are mixed one or more infrared radiating materials selected from silica, germanium, compound semiconductors and metal oxides such as tourmaline and composite carbon particles having an SP³ diamond structure and an SP² graphite structure formed by shockwaves, the composite powder being mixed with or affixed to the fiber, and having an infrared radiation function and a charged particle emission function.

15. The functional fiber recited in claim 14, wherein the composite powder comprises no less than 2 wt % of the composite carbon particles having an SP³ diamond structure and an SP² graphite structure formed by shockwaves.

16. A functional thread made using the functional fiber recited in claim 14 or 15.

17. A processed product or formed product comprising the functional fiber recited in claim 14 or 15 or the functional thread recited in claim 16.
